(19) United States
(12) Patent Application Publication
(10) Pub. No.: US 2006/0234265 A1
(43) Pub. Date: Oct. 19, 2006

(54) Microarrays having multi-functional, compartmentalized analysis areas and methods of use

(76) Inventors: Jim Richey, Sheldon, SC (US); Timothy M. Londergan, Seattle, WA (US); Dan L. Jin, Bothell, WA (US)

Correspondence Address: FISH & RICHARDSON P.C., PO BOX 1022, MINNEAPOLIS, MN, 55440-1022, US

(21) Appl. No.: 11/375,885

(22) Filed: Mar. 15, 2006

Related U.S. Application Data
(60) Provisional application No. 60/663,932, filed on Mar. 21, 2005.

(51) Int. Cl. C12Q 1/68; G01N 33/53; C12M 1/34

(52) U.S. Cl. 435/6; 435/7.5; 435/287.2

(57) ABSTRACT

Microarrays are provided that include multiple analysis areas. Each analysis area can include first and second active areas, and first and second hydrophobic areas. The first hydrophobic area surrounds the first active area, and the second active area surrounds the first hydrophobic area. The microarrays are useful in analytical chemistry, biochemistry and biology.

### CROSS-REFERENCE TO RELATED APPLICATIONS

This application claims priority from U.S. Provisional Application Ser. No. 60/663,932, filed Mar. 21, 2005.

### BACKGROUND OF THE INVENTION

All patents, patent applications, and publications cited within this application are incorporated herein by reference to the same extent as if each individual patent, patent application, or publication was specifically and individually incorporated by reference.

The invention relates generally to microarrays used in analytical chemistry, biochemistry, and biology. Typical microarrays include localized areas variously identified as, for example, “defined regions,” “spots,” “addresses,” “pads” or “wells,” often arranged in row and columns. Spots usually contain a chemical substance (e.g., a ligand) immobilized on a surface. The chemical substance typically is of known composition and is capable of binding or somehow reacting with an analyte (i.e., a substance of interest) to localize the analyte in a particular spot. Spots may be isolated from other spots by physical barriers such as ridges or hydrophobic barriers such as polymer films. In some cases, further reaction of the analyte is carried out in the spot. The bound analyte may be probed using techniques such as fluorescence imaging or mass spectrometry. Microarrays can enable high throughput analysis of complex mixtures containing biologically interesting analytes, particularly when each spot is individually addressable. One of the main uses of microarrays has been in the area of genomics, where long strands of DNA can be identified by analyzing the binding of shorter, complementary DNA fragments to oligonucleotide microarrays. Other microarrays include protein microarrays that have been used, for example, in proteomics (see, Curr. Opin. Chem. Biol. 2004, 8(1), 8). However, spots typically have only one function (e.g., immobilizing a DNA having a specific nucleotide sequence), which limits what can be done to the analyte and the amount of information available in the analysis. What is needed are microarrays and methods of use that enable multi-functional spots for simultaneous or serial probing (e.g., fluorescence probing of binding and mass spectrometric determination of composition) without problems associated with the multi-functional components reacting with each other or interfering with binding of the analyte (by, for example, reacting with the immobilized ligand or the analyte).

### SUMMARY OF THE INVENTION

A microarray is described that comprises a plurality of multi-functional, compartmentalized analysis areas. The microarray comprises a plurality of analysis areas, wherein each analysis area comprises: a first active area; a first hydrophobic area; a second active area; and a second hydrophobic area, wherein the first hydrophobic area surrounds the first active area and the second active area surrounds the first hydrophobic area. The first active area and the second active area of each spot may be used individually at each occurrence, for example to bind and/or modify an analyte of interest. The first hydrophobic area separates the first active area from the second active area and this allows the two areas to be isolated, or compartmentalized, from each other. In one embodiment, the first hydrophobic area and the second hydrophobic area each comprises a self-assembled monolayer. The first hydrophobic area, the second active area, and the second hydrophobic area can be concentric rings. In one embodiment, the first active area comprises an immobilized polypeptide. The immobilized polypeptide may comprise an antigen or an antibody. A “polypeptide” is any chain of amino acids, regardless of length or post-translational modification (e.g., glycosylation or phosphorylation). The term “antibody” includes all classes of immunoglobulins, and further includes both polyclonal and monoclonal antibodies as well as chimeric and/or humanized antibodies. The term “antibody” further includes functional fragments of whole immunoglobulin molecules, such as Fab, F(ab′)2 and Fc fragments. In another embodiment, the first active area comprises immobilized avidin, immobilized non-glycosylated avidin, or immobilized streptavidin. In yet another embodiment, the first active area comprises an immobilized polynucleotide, for example a cDNA. In some embodiments, the first active area further comprises an immobilized fusion tag ligand. In other embodiments, the second active area comprises an immobilized polypeptide, for example an immobilized enzyme. In some embodiments, the enzyme is a proteolytic enzyme. In another embodiment, each analysis area comprises: a) a first active area; a first hydrophobic area; a second active area; and a second hydrophobic area, wherein the first hydrophobic area surrounds the first active area and the second active area surrounds the first hydrophobic area; b) the first hydrophobic area and the second hydrophobic area each comprises a self-assembled monolayer; c) the first hydrophobic area, the second active area, and the second hydrophobic area are concentric rings; d) the first active area comprises an immobilized cDNA and a fusion tag ligand; and e) the second active area comprises an immobilized proteolytic enzyme.

Another embodiment is a method comprising: a) providing a microarray comprising a plurality of analysis areas, wherein each analysis area comprises: a first active area comprising an immobilized polypeptide; a first hydrophobic area; a second active area comprising an immobilized proteolytic enzyme; and a second hydrophobic area, wherein the first hydrophobic area surrounds the first active area and the second active area surrounds the first hydrophobic area; b) forming a complex between a target polypeptide in a first solution and the immobilized polypeptide in the first active area of at least one of the analysis areas; c) dissociating the target polypeptide from the surface into a second solution having a volume that is isolated to the analysis area where the complex was formed; d) increasing the volume of the second solution to such an extent that spillover into the second analysis area occurs; and e) digesting the target polypeptide with the immobilized proteolytic enzyme. In one embodiment, the first solution is flooded onto the microarray surface. The first solution can be dispensed (or “printed”) into the first active area. Preferably, the volume of the second solution is confined substantially over the second active area after spillover occurs. The first hydrophobic area, the second active area, and the second hydrophobic area can be concentric rings. Dissociating the target polypeptide from the surface may be accomplished, for example, by detaching the whole complex from the surface by detaching the immobilized polypeptide from the surface, or by dissociating the complex of the target polypeptide and the immobilized polypeptide (thereby leaving the immobilized polypeptide attached to the surface). In some embodiments, the immobilized polypeptide comprises an antigen. In other embodiments, the immobilized polypeptide comprises an antibody. The proteolytic enzyme can be a serine protease, for example a trypsin, chymotrypsin, or elastase.

In another embodiment, a method comprises: a) providing a microarray comprising a plurality of analysis areas, wherein each analysis area comprises: a first active area comprising an immobilized cDNA and an immobilized fusion tag ligand; a first hydrophobic area; a second active area comprising an immobilized proteolytic enzyme; and a second hydrophobic area, wherein the first hydrophobic area surrounds the first active area and the second active area surrounds the first hydrophobic area; b) forming an aqueous solution comprising cell-free protein expression machinery on the first active area; c) expressing a fusion protein that binds to the fusion tag ligand, the fusion protein corresponding to the cDNA; d) forming a complex between a target polypeptide in a first solution and the immobilized fusion protein in the first active area of at least one of the analysis areas; e) dissociating the target polypeptide from the surface into a second solution having a volume that is isolated to the analysis area where the complex was formed; f) increasing the volume of the second solution until spillover into the second analysis area occurs; and g) digesting the target polypeptide with the proteolytic enzyme. In another embodiment, the method further comprises performing a chemical analysis on the complex before dissociating the target polypeptide. The analysis may comprise, for example, spectroscopy, fluorescence, or surface plasmon resonance. The chemical analysis may involve, for example, adding an antibody to recognize the complexed target polypeptide or the complex itself. In one embodiment, the method further comprises performing mass spectrometry after digesting the target polypeptide. In another embodiment, the mass spectrometry is MALDI.

Other embodiments include a method comprising: a) providing a microarray comprising a plurality of analysis areas, wherein each analysis area comprises: a first active area comprising an immobilized capture agent; a first hydrophobic area; a second active area comprising an immobilized reactive group; and a second hydrophobic area, wherein the first hydrophobic area surrounds the first active area and the second active area surrounds the first hydrophobic area; b) forming a complex between an analyte in a first solution and the immobilized capture agent in the first active area of at least one of the analysis areas; c) dissociating the analyte from the surface into a second solution having a volume that is isolated to the analysis area where the complex was formed; d) increasing the volume of the second solution until spillover into the second analysis area occurs; and e) reacting the analyte with the immobilized reactive group. In many embodiments, the volume of the second solution is confined substantially over the second analysis area after spillover occurs. In one embodiment, the first hydrophobic area is a self-assembled monolayer, the second hydrophobic area comprises a self-assembled monolayer, the immobilized capture agent comprises a polypeptide, the immobilized reactive group comprises an enzyme, and the analyte comprises a polypeptide. In other embodiments, the immobilized capture agent is a polypeptide, the analyte is a polypeptide, and the immobilized reactive group is a proteolytic enzyme.

In other embodiments, a method comprises: a) forming a complex between an analyte and a capture agent in a first area of a surface that is surround by a first hydrophobic area; b) dissociating the analyte into a solution that is confined substantially over the first area; c) increasing the volume of the solution until spillover into a second area of the surface occurs, wherein the second area surrounds the first hydrophobic area, is surrounded by a second hydrophobic area, and comprises an immobilized reactive group; d) and reacting the analyte with the immobilized reactive group. In many embodiments, the first area and the second area are hydrophilic and the first hydrophobic area and the second hydrophobic area each comprises a self assembled monolayer. In one embodiment, the immobilized capture agent is a polypeptide, the analyte is a polypeptide, and the immobilized reactive group is a proteolytic enzyme.

### DETAILED DESCRIPTION

A microarray is described that comprises multi-functional analysis areas. The multi-functional analysis areas are compartmentalized. What is meant by “analysis area” is a localized portion or section of the microarray where, for example, a particular analyte may be bound in one compartment (e.g. an active area within the analysis area) and then serially modified in another compartment (e.g. another active area within the analysis area) for further analysis. Multiple analysis areas can be arranged, for example, in rows and columns to form the microarray. The pattern of the multiple analysis areas, as well as the pattern of active areas within each analysis area, may be formed by methods known to those skilled in the art including photolithography, printing, and stamping, for example see U.S. Pat. No. 6,565,813. In many embodiments, the microarray comprises a plurality of analysis areas, wherein each analysis area comprises: a first active area; a first hydrophobic area; a second active area; and a second hydrophobic area, wherein the first hydrophobic area surrounds the first active area and the second active area surrounds the first hydrophobic area. The first active area and the second active area of each analysis area may be used individually at each occurrence, for example, to bind and/or modify an analyte of interest. The first hydrophobic area separates the first active area from the second active area, thus allowing the two areas to be isolated, or compartmentalized, from each other. This isolation can be crucial, for example, in a process that immobilizes a polypeptide in the first active area, non-destructively probes the polypeptide by surface plasmon resonance, digests the polypeptide in the second active area, and probes the digestion product with mass spectrometry. The hydrophobic areas may be formed from materials that generally lack polar groups at the surface and thus lack the ability to form strong interactions (e.g., hydrogen bonds) with water. Such materials may include thin films made from generally non-polar polymers such as poly(tetrafluoro)ethylene or from thin layers of small molecules having non-polar groups. Hydrophobic polymers and small molecules may be deposited on a surface by a number of different methods including spin coating, dip coating, and painting, all of which may be used alone or in combination with other techniques like photolithography and dry etching. In one example, the hydrophobic areas maybe formed by depositing a hydrophobic polymer or small molecule in photoresist free areas on a photolithographically patterned surface. In other cases, the polymer or small molecule may be deposited on the whole surface and then etched away in the areas that correspond to the first and second active areas. The hydrophobic polymer may also be, for example, a photoresist. The hydrophobicity of an area can be determined by measuring one or more contact angles formed by a water droplet on the hydrophobic surface. In one embodiment, the first hydrophobic area and the second hydrophobic area each comprises a self-assembled monolayer. Self-assembled monolayers (SAMs) are typically formed from a molecule having a hydrophobic alkyl chain and a functional group (e.g., a thiol or trialkoxysilane) that can react with a surface (e.g., gold or glass). The hydrophobic alkyl chain may further include substituents such as fluorine. The functional group of a SAM forming molecule is attached to a particular surface and the hydrophobic alkyl chain extends from the surface and interacts with neighboring alkyl chains to form a relatively ordered single layer having a thickness approximately or less than the length of an individual SAM forming molecule. The SAM may be formed by a variety of methods including flooding a surface with the SAM forming molecule and stamping, for example see U.S. Pat. No. 5,512,131. In many embodiments, the first active area is hydrophilic. In other embodiments, the second active area is hydrophilic. In one embodiment, the first active area comprises a gold thin film.

In one embodiment, the first active area comprises an immobilized polypeptide. The surface to which the polypeptide is immobilized can be any one of those known to the skilled artisan, including, for example, silicon oxide, glass, or gold. The polypeptide can be immobilized on the surface through methods known to those skilled in the art that use covalent linkages, non-covalent linkages, or a combination of both. In some cases, these linkages are accomplished by using “crosslinkers” that have one functional group that can react with or bind to a surface and another functional group that can react with or bind to a polypeptide. The crosslinkers may use groups that covalently react, non-covalently bind, or a combination of both. The immobilized polypeptide may comprise an antigen or an antibody. In another embodiment, the first active area comprises immobilized avidin, immobilized non-glycosylated avidin, or immobilized streptavidin. The avidin may be immobilized directly on the surface, or can be immobilized by, for example, a crosslinker or by biotin that is linked to the surface, or by other methods. The use and utility of antigens, antibodies, and avidins in binding of targets is well described in the art.

In another embodiment, the first active area comprises an immobilized polynucleotide, for example an oligonucleotide or longer polynucleotide such as a cDNA. The polynucleotide can be immobilized on the surface by any number of methods known in the art that include covalent linkages, noncovalent linkages, or a combination of both. In many embodiments, the immobilized oligonucleotide is a cDNA. The immobilized polynucleotide can be introduced to the first active area, for example, in small aqueous volumes by methods known to those skilled in the art. In some embodiments, the first active area further comprises an immobilized fusion tag ligand. Fusion tag ligands are known in the art and bind to fusion tags that are covalently bonded to polypeptides. A polypeptide that includes a covalently bonded fusion tag can be, for example, a fusion protein. Some common fusion tag ligands/fusion tags are, for example, glutathione/glutathione-S-tranferase, chitin/chitin binding protein, cellulose/celluslase, maltose or dextrin/maltose binding protein, methotrexate/dihyrofolate reductase, FK506/FKBP, and chelated nickel or cobalt/polyhistidine (6×His). The fusion tag ligand may comprise, for example, a polypeptide of 5 to 55 amino acids and the fusion tag may comprise a polypeptide of 5 to 55 amino acids. In one embodiment, the fusion tag ligand and the fusion tag comprise polypeptides that form a coiled-coil dimer. Coiled-coil dimers are known in the art and include fusion tag ligands (e.g., Jun) that bind to the heptad repeat region of fusion tags with heptad repeat regions (e.g., Fos); for example, see Science 2004, 305, 86. The coiled-coil dimers may be homodimers or heterodimers. In one embodiment, the fusion tag ligand is bound to the fusion tag of a fusion protein. In many embodiments, the fusion protein is encoded by the immobilized cDNA. In these cases, the fusion protein may be expressed from the immobilized cDNA using cell-free expression methods that are known in the art. Cell-free expression methods include those, for example, found in U.S. Pat. No. 6,800,453; US Pat Appl 2004/0161748; Nucleic Acids Res. 2001, 29(15), e73; or Science 2004, 305, 86. In some embodiments, the fusion protein expressed from the immobilized cDNA may comprise, for example, an antigen or an antibody.

In many embodiments, the second active area comprises an immobilized polypeptide. In one embodiment, the immobilized polypeptide of the second active area comprises an enzyme. In some embodiments, the enzyme is a proteolytic enzyme (e.g., a serine protease). Examples of serine proteases include trypsin, chymotrypsin, or elastase. Proteolytic enzymes are known in the art and can be used to digest polypeptides. The digestion of polypeptides is useful in analytical techniques such as, for example, MALDI. Proteolytic enzymes with site-specific activity can be used in combination to provide information about the polypeptide sequence.

The number of analysis areas, the size of the analysis areas, and the configurations of the first hydrophobic area, the second active area, and the second hydrophobic area may vary depending on the application and what is desirable by the user. In one embodiment, the first hydrophobic area, the second active area, and the second hydrophobic area are concentric rings. The size of the analysis area may range, for example, from 50² μm to 50,000² μm. In many embodiments, the number of analysis areas is, for example, 500-500,000. The analysis areas may be arranged in columns and rows.

In one embodiment, each analysis area comprises: a) a first active area; a first hydrophobic area; a second active area; and a second hydrophobic area, wherein the first hydrophobic area surrounds the first active area and the second active area surrounds the first hydrophobic area; b) the first hydrophobic area and the second hydrophobic area each comprises a self-assembled monolayer; c) the first hydrophobic area, the second active area, and the second hydrophobic area are concentric rings; d) the first active area comprises an immobilized cDNA and a fusion tag ligand; and e) the second active area comprises an immobilized proteolytic enzyme.

Another embodiment is a method comprising: a) providing a microarray comprising a plurality of analysis areas, wherein each analysis area comprises: a first active area comprising an immobilized polypeptide; a first hydrophobic area; a second active area comprising an immobilized proteolytic enzyme; and a second hydrophobic area, wherein the first hydrophobic area surrounds the first active area and the second active area surrounds the first hydrophobic area; b) forming a complex between a target polypeptide in a first solution and the immobilized polypeptide in the first active area of at least one of the analysis areas; c) dissociating the target polypeptide from the surface into a second solution having a volume that is isolated to the analysis area where the complex was formed; d) increasing the volume of the second solution to such an extent that spillover into the second analysis area occurs; and e) digesting the target polypeptide with the immobilized proteolytic enzyme. The immobilized polypeptide and the immobilized proteolytic enzyme may be attached to the surface as described above. A skilled artisan would appreciate that forming a complex between a target polypeptide and the immobilized polypeptide may be performed under conditions that are compatible with and favor complex formation between the particular target polypeptide and immobilized polypeptide. The first solution may be, for example, a buffer solution made from purified target polypeptides, plasma taken directly from lysed cells, or bodily fluids taken directly from a patient in a clinical setting. In one embodiment, the first solution is flooded onto the microarray surface. In another embodiment, the first solution is printed into the first active area, as in known in the art. Dissociating the target polypeptide may involve having a reagent or a number of different reagents in the second solution. The reagent or reagents needed to dissociate the target polypeptide will vary depending on the particular target polypeptide and immobilized polypeptide. The reagents may, for example, alter the pH, alter the ionic strength, act as a chaotrope (e.g., urea or guanidine hydrochloride), or act as a displacing ligand. Increasing the volume of the second solution may be accomplished, for example, by printing a liquid onto the second solution. The amount of volume that is needed to cause spillover into the second active area will vary depending, for example, on the size of the first active area, the size of the first hydrophobic area, the hydrophobicity of the first hydrophobic area, and the viscosity of the second solution. In one embodiment, the volume of the second solution is confined substantially over the second active area after spillover occurs. In many embodiments, the first hydrophobic area and the second hydrophobic area each comprises a self-assembled monolayer. The number of analysis areas, the size of the analysis areas, and how the analysis areas are arranged may be as described above. In one embodiment, the first hydrophobic area, the second active area, and the second hydrophobic area are concentric rings. In many embodiments, the first active area comprises a gold thin film.

Dissociating the target polypeptide from the surface may be accomplished, for example, by detaching the whole complex from the surface by detaching the immobilized polypeptide from the surface, or by dissociating the complex of the target polypeptide and the immobilized polypeptide (thereby leaving the immobilized protein attached to the surface). In some embodiments, the immobilized polypeptide comprises an antigen. In other embodiments, the immobilized polypeptide comprises an antibody. In one embodiment, the proteolytic enzyme comprises a serine protease. Examples of serine proteases include trypsin, chymotrypsin, or elastase. The conditions used to digest the target polypeptide will vary depending, for example, on the particular serine protease, the particular target polypeptide, and the amount of digestion desired. The digestion of polypeptides with serine proteases is well described in the art.

In one embodiment, a method comprises: a) providing a microarray comprising a plurality of analysis areas, wherein each analysis area comprises: a first active area comprising an immobilized cDNA and an immobilized fusion tag ligand; a first hydrophobic area; a second active area comprising an immobilized proteolytic enzyme; and a second hydrophobic area, wherein the first hydrophobic area surrounds the first active area and the second active area surrounds the first hydrophobic area; b) forming an aqueous solution comprising cell-free protein expression machinery on the first active area; c) expressing a fusion protein that binds to the fusion tag ligand, the fusion protein being encoded by the cDNA; d) forming a complex between a target polypeptide in a first solution and the immobilized fusion protein in the first active area of at least one of the analysis areas; e) dissociating the target polypeptide from the surface into a second solution having a volume that is isolated to the analysis area where the complex was formed; f) increasing the volume of the second solution until spillover into the second analysis area occurs; and g) digesting the target polypeptide with the proteolytic enzyme. The cell-free expression machinery will vary depending on the vector and conditions that are used, and may be any of those, for example, described in U.S. Pat. No. 6,800,453; US Pat Appl 2004/0161748; Nucleic Acids Res. 2001, 29(15), e73; or Science 2004, 305, 86. The immobilized cDNA, fusion protein, the fusion tag ligand, and proteolytic enzyme may be as described above.

In another embodiment, the method further comprises performing a chemical analysis on the complex before dissociating the target polypeptide. The analysis may comprise, for example, spectroscopy, fluorescence, or surface plasmon resonance, and may involve, for example, adding an antibody to recognize the complexed target polypeptide or the complex itself The method can further comprise performing mass spectrometry (e.g., MALDI) after digesting the target polypeptide.

Other embodiments include a method comprising: a) providing a microarray comprising a plurality of analysis areas, wherein each analysis area comprises: a first active area comprising an immobilized capture agent; a first hydrophobic area; a second active area comprising an immobilized reactive group; and a second hydrophobic area, wherein the first hydrophobic area surrounds the first active area and the second active area surrounds the first hydrophobic area; b) forming a complex between an analyte in a first solution and the immobilized capture agent in the first active area of at least one of the analysis areas; c) dissociating the analyte from the surface into a second solution having a volume that is isolated to the analysis area where the complex was formed; d) increasing the volume of the second solution until spillover into the second analysis area occurs; and e) reacting the analyte with the immobilized reactive group. In many embodiments, the volume of the second solution is confined substantially over the second analysis area after spillover occurs. In one embodiment, the first hydrophobic area is a self-assembled monolayer, the second hydrophobic area comprises a self-assembled monolayer, the immobilized capture agent comprises a polypeptide, the immobilized reactive group comprises an enzyme, and the analyte comprises a polypeptide. In other embodiments, the immobilized capture agent is polypeptide, the analyte is a polypeptide, and the immobilized reactive group is a proteolytic enzyme.

In other embodiments, a method comprises: a) forming a complex between an analyte and a capture agent in a first area of a surface that is surround by a first hydrophobic area; b) dissociating the analyte into a solution that is confined substantially over the first area; c) increasing the volume of the solution until spillover into a second area of the surface occurs, wherein the second area surrounds the first hydrophobic area, is surrounded by a second hydrophobic area, and comprises an immobilized reactive group; d) and reacting the analyte with the immobilized reactive group. In many embodiments, the first area and the second area are hydrophilic and the first hydrophobic area and the second hydrophobic area each comprises a self assembled monolayer. In one embodiment, the immobilized capture agent is a polypeptide, the analyte is a polypeptide, and the immobilized reactive group is a proteolytic enzyme.

Other embodiments are within the scope of the following claims.

What is claimed is:

1. A microarray comprising a plurality of analysis areas, wherein each analysis area comprises: a first active area; a first hydrophobic area; a second active area; and a second hydrophobic area, wherein the first hydrophobic area surrounds the first active area and the second active area surrounds the first hydrophobic area.

2. The microarray of claim 1, wherein the first hydrophobic area and the second hydrophobic area each comprises a self-assembled monolayer.

3. The microarray of claim 2, wherein the first active area is hydrophilic.

4. The microarray of claim 2, wherein the first active area comprises an immobilized polypeptide.

5. The microarray of claim 4, wherein the immobilized polypeptide comprises an antigen.

6. The microarray of claim 4, wherein the immobilized polypeptide comprises an antibody.

7. The microarray of claim 2, wherein the first active area comprises immobilized avidin, immobilized non-glycosylated avidin, or immobilized streptavidin.

8. The microarray of claim 2, wherein the first active area comprises an immobilized oligonucleotide.

9. The microarray of claim 8, wherein the oligonucleotide is a cDNA.

10. The microarray of claim, 9, wherein the first active area further comprises an immobilized fusion tag ligand.

11. The microarray of claim 10, wherein the fusion tag ligand is bound to the fusion tag of a fusion protein.

12. The microarray of claim 11, wherein the fusion tag ligand comprises glutathione, chitin, cellulose, maltose, dextrin, methotrexate, FK506, chelated nickel, or chelated cobalt.

13. The microarray of claim 11, wherein the fusion tag ligand comprises a polypeptide of 5 to 55 amino acids and the fusion tag comprises a polypeptide of 5 to 55 amino acids.

14. The microarray of claim 13, wherein the fusion tag ligand and the fusion tag are a coiled-coil.

15. The microarray of claim 11, wherein the fusion protein is complementary to the immobilized cDNA.

16. The microarray of claim 15, wherein the fusion protein expressed from the immobilized cDNA comprises an antigen.

17. The microarray of claim 15, wherein the fusion protein expressed from the immobilized cDNA comprises an antibody.

18. The microarray of claim 3, wherein the second active area is hydrophilic.

19. The microarray of claim 3, wherein the second active area comprises an immobilized polypeptide.

20. The microarray of claim 19, wherein the immobilized polypeptide of the second active area comprise an enzyme.

21. The microarray of claim 20, wherein the enzyme is proteolytic.

22. The method of claim 21, wherein the enzyme comprises a serine protease.

23. The microarray of claim 22, wherein the enzyme comprises trypsin, chymotrypsin, or elastase.

24. The microarray of claim 1, wherein the first hydrophobic area, the second active area, and the second hydrophobic area are concentric rings.

25. The microarray of claim 1, wherein the analysis area is 50² μm to 50,000² μm.

26. The microarray of claim 1, where the number of analysis areas is 500-500,000.

27. The microarray of claim 26, wherein the analysis areas are arranged in columns and rows.

28. The microarray of claim 1, wherein the first active area comprises a gold thin film.
